(19) United States
(12) Patent Application Publication
(10) Pub. No.: US 2003/0114482 A1
(43) Pub. Date: Jun. 19, 2003

(54) Use of retinoid receptor antagonists or agonists in the treatment of cartilage and bone pathologies

(76) Inventors: Maurizio Pacifici, Swarthmore, PA (US); Roshantha A. Chandraratna, Laguna Hills, CA (US)

Correspondence Address: Allergan Inc, 2525 Dupont Drive, Irvine, CA, 92612, US

(21) Appl. No.: 09/552,823

(22) Filed: Apr. 20, 2000

Related U.S. Application Data
(63) Continuation-in-part of application No. 09/464,344, filed on Dec. 15, 1999, now Pat. No. 6,313,168.

(51) Int. Cl. A61K 31/47; A61K 31/382; A61K 31/353; A61K 31/192

(52) U.S. Cl. 514/311; 514/432; 514/456; 514/570

(57) ABSTRACT

The present invention relates to methods for treating cartilage and bone pathologies, including bone growth related diseases such as osteoarthritis or osteoporosis, comprising administering therapeutically effective amounts of retinoid receptor antagonists or retinoid receptor agonists.

### CROSS REFERENCE TO RELATED APPLICATIONS

This application is a continuation-in-part of Ser. No. 09/464,344, filed Dec. 15, 1999.

### BACKGROUND OF THE INVENTION

Articular cartilage is a unique tissue present in the joints in the limbs, trunk and cervical region. The tissue is composed of articular chondrocytes and an abundant extracellular matrix that contains several well characterized macromolecules, including proteoglycan aggregates, hyaluronic acid, link protein and type II collagen fibrils. The chondrocytes are responsible for the synthesis, deposition and maintenance of the matrix components. The proteoglycan aggregates are large supramolecular structures that bind large quantities of water molecules and ions and provide the tissue with bioelasticity. The collagen fibrils form a three dimensional network that is able to withstand tensile and shear forces and provides the tissue with tensile strength. Together, the proteoglycan aggregates and collagen fibrils are responsible for a fundamental biomechanical property of articular cartilage, resilience. This property allows the tissue to undergo reversible changes in shape and volume that result from physical forces acting on the joints during movement, and thus permit normal functioning of the joints. Under normal healthy circumstances, articular chondrocytes remain active and phenotypically stable throughout life; in turn, this allows articular cartilage to maintain its structural and organization characteristics and to perform its biomechanical roles in the joints throughout life.

Endochondral ossification is the process by which the cartilaginous skeletal elements present in the embryo and growing organism are replaced by definitive bone elements. The process starts in the second half of embryogenesis and is concluded at the end of puberty when skeletal growth ceases. Endochondral ossification is a highly-regulated multistep process that involves several distinct steps of chondrocyte maturation and is best appreciable in long bone growth plates in the limbs. During endochondral ossification, resting immature chondrocytes first undergo a phase of rapid cell proliferation. The cells then withdraw from the cell cycle and enter a phase of active matrix production. Matrix components synthesized at this step are typical cartilage matrix macromolecules, including proteoglycans (aggrecan), type II collagen, link protein and hyaluronan. The postmitotic matrix-synthesizing cells then begin to enlarge in size and change from flat to oval-round in shape. This step is called the pre-hypertrophic stage and is characterized by synthesis of new proteins, including the signaling factor Indian hedgehog. The cells continue to enlarge and advance to their ultimate stage of maturation, the hypertrophic stage. The biosynthetic repertoire of hypertrophic chondrocytes changes dramatically, and the cells initiate production of various new proteins including: metalloproteases, type X collagen, alkaline phosphatase and annexin V-rich matrix vesicles. As they undergo these changes in biosynthesis, the hypertrophic chondrocytes also begin synthesis of bone-characteristic type I and III collagens and deposit apatite crystals in the matrix, thus transforming hypertrophic cartilage into a bone-like tissue. Finally, they undergo apoptosis. As a result, the tissue becomes amenable to invasion by bone and bone marrow precursor cells, which then proceed to remove the hypertrophic tissue and replace it with definitive bone tissue.

A large number of studies have been carried out during the last several years to identify and characterize the mechanisms regulating endochondral ossification. Interest in these mechanisms reflects the fact that defects in endochondral ossification are associated, and probably cause, congenital and acquired conditions of skeletogenesis (Jacenko et al., J. Rheumatol. 22:3941 (1995)). Interestingly, several molecules have been shown to have a negative role in endochondral ossification and to limit the rates at which chondrocytes progress from the immature to the hypertrophic stage. These molecules include fibroblast growth factor-2 (FGF-2), fibroblast growth factor receptor-3 (FGF-R3), parathyroid-related protein (PTH-rP), and Indian hedgehog (IHH) (Coffin, et al., Mol. Biol. Cell, 6:1861-1873 (1995); Colvin et al., Nature Genet., 12:390-397 (1996); Vortkamp et al., Science, 273:613-622 (1996)). However, very few positive factors have been identified to date, which would have the critical role of counteracting the negative factors and allow the endochondral process to advance and reach its conclusion.

Pathologies associated with bone growth include osteoarthritis. Osteoarthritis is a degenerative disease of the joints that causes progressive loss of articular tissue. The disease, for which presently no cure or effective treatment exists, affects over 10% of the population over 60 years of age. Osteoarthritis is probably initiated by a number of factors, including mechanical insults derived from life-long use of the joints. Once articular cartilage is damaged, the disease progresses and numerous changes occur in the cells and matrix. At sites most affected by the disease, the articular chondrocytes can reinitiate proliferation and begin to acquire abnormal phenotypic traits. These include synthesis of type I and III collagens, cell hypertrophy, type X collagen synthesis, alkaline phosphatase activity increased proteolytic activity and even matrix mineralization (Hamerman, New Engl. J. Med. 320, 1322-1330 (1989); Nerlich, et al., Vichows Archiv. B. Cell Pathol. 63, 249-255 (1993); von der Mark, K. et al., Acta Orthop. Scand. 266, 125-129 (1995)). At the same time, while synthesis of proteoglycans increases, net proteoglycan content decreases because of increased matrix degradation by metalloproteases and other degradative enzymes. There are also reports that the articular chondrocytes can display signs of cellular degeneration and apoptosis. Once the articular cells disappear and the matrix degenerates, the tissue is replaced by non-functional scar tissue or even bony tissue.

Thus, a need exists for effective therapeutic methods for the treatment of cartilage and bone pathologies, including bone growth related diseases such as osteoarthritis.

### SUMMARY OF THE INVENTION

The present invention provides a method for treating a cartilage or bone pathology comprising administering a therapeutically effective amount of a retinoid receptor antagonist. According to one preferred embodiment, the retinoid receptor antagonist is an RAR receptor antagonist, and preferably an RAR alpha, beta, or gamma receptor antagonist.

The present invention further provides a method for treating a cartilage or bone pathology comprising antagonizing RARγ receptors associated with the pathology.

In a further embodiment, the present invention provides a method for ameliorating the symptoms associated with cartilage and bone pathologies comprising administering a therapeutically effective amount of a retinoid receptor antagonist.

The invention additionally provides a method for treating a cartilage or bone pathology comprising administering a therapeutically effective amount of a pharmaceutical composition comprising a retinoid receptor antagonist and a pharmaceutically acceptable carrier or excipient.

The present invention further provides a method for enhancing cartilage or bone growth comprising administering a therapeutically effective amount of a pharmaceutical composition comprising a retinoid receptor agonist.

In a further embodiment, the present invention provides a method for stimulating osteoprogenitor cells and osteoblasts comprising administering a therapeutically effective amount of a retinoid receptor agonist.

The invention additionally provides a method for enhancing cartilage or bone growth comprising administering a therapeutically effective amount of a pharmaceutical composition comprising a retinoid receptor agonist and a pharmaceutically acceptable carrier or excipient.

### DETAILED DESCRIPTION OF THE INVENTION

The present invention provides a method of treating cartilage and bone pathologies, including bone growth related diseases, comprising the use of retinoid receptor antagonists. Bone growth related diseases include those involving pathological ossification such as osteoarthritis, multiple cartilaginous exostoses and osteoblastic tumors including osteoid osteoma, osteosarcoma and osteoma; and osteitis deformans (see generally, Pathological Basis of Disease, Robbins, et al. W. B. Saunders Co. (1979)). At the molecular level retinoids exert their biological effects through two families of nuclear receptors, retinoic acid receptors (RARs) and retinoid X receptors (RXRs), which belong to the superfamily of steroid/thyroid/vitamin D3 nuclear receptors.

RARs and RXRs are ligand-dependent transcription factors which regulate gene expression in at least two different ways: (a) they upregulate the expression of genes by binding to the RA-responsive elements (RAREs) present in their promoters or (b) they down-regulate the expression of genes by antagonizing the enhancer action of certain other transcription factors, such as AP1. The distinct isotypes of RARs (α, β and γ) and RXRs (α, β and γ) are encoded by six separate genes. Each RAR isotype is further expressed as several isoforms differing in their N-terminal A region, which are generated by alternative splicing and/or by differential usage of more than one promotor. RARα is expressed as two main isoforms (α1 and α2). RARβ as four isoforms (β1, β2, β4 and β4) and RARγ as two main isoforms (γ1 and γ2). RARs are believed to function exclusively in vivo as RAR-RXR heterodimers.

It has been found that hypertrophic chondrocytes present in long bone models in the developing limb express high levels of RAR, specifically RARγ, and contain endogenous retinoids. As described in detail in the Examples, to determine the roles of RARγ and endogenous retinoids, beads filled with retinoid antagonist AGN 109 were placed in the vicinity of the developing long bone models at early stages of chick embryo development. The embryos were then reincubated in the presence of RARγ antagonist and the effects of antagonist treatment determined at various time points. It was found that chondrocyte maturation and long bone development are interrupted by antagonist treatment. In control limbs, the long bone models contained hypertrophic chondrocytes in their central portions (called the diaphysis) that synthesized type X collagen, alkaline phosphatase, and were mineralizing their matrix. Moreover, the hypertrophic cartilage was undergoing invasion by bone and marrow precursor cells and active bone deposition. In sharp contrast, the retinoid antagonist-treated long bones were entirely cartilaginous and contained no hypertrophic chondrocytes, type X collagen or alkaline phosphatase. In addition, calcium deposition and bone formation was not observed in the test group. Thus, retinoids are positive regulators of endochondral ossification, and appear to interfere with normal retinoid signaling by treatment with retinoid antagonists which blocks chondrocyte maturation and endochondral ossification (see also, Koyama et al., Develop. Biol. 208(2): 375-391 (1999)).

Accordingly, the present invention provides methods for interrupting or even reversing the acquisition of growth plate-like traits by articular chondrocytes during osteoarthritis or other conditions of articular cartilage leading to calcium deposition. Articular chondrocytes are those chondrocytes located in the skeletal joints. Thus, suitable retinoid receptor antagonists should prevent (a) hypertrophy of the cells, (b) expression of metalloproteases and alkaline phosphatase activity, (c) mineral deposition and even apoptosis, and (d) switches in collagen types, all of which occur in articular chondrocytes during the disease process. By preventing or slowing down such phenotypic changes, the antagonists should permit articular chondrocytes to carry out more effective repair of the matrix and tissue and may cause cessation of the degenerative process. The methods of the present invention are not linked to effecting articular chondrocytes but may be used to effect chondrocytes at any location in the skeletal system and associated with any phase of skeletal development or bone growth related pathology.

Any retinoid receptor antagonist presently known in the art, or subsequently developed, may be used in practicing the claimed methods. The synthesis of exemplary receptor antagonists is described, by way of example only, in U.S. Pat. Nos. 5,877,207; 5,514,825; 5,648,514; 5,728,846; 5,739,338; 5,760,276; 5,776,699; 5,773,594; 5,763,635; and 5,808,124 and U.S. Ser. Nos. 08/840,040 and 08/845,019, incorporated herein by reference in their entireties.

In a preferred method, the antagonist is an RAR antagonist, and more preferably an RARaβγ antagonist. However, antagonists with activity specific for a particular isotype and/or isoform or a combination thereof may also be used in the present methods. Thus, antagonists specific for RARa, β, γ or combinations thereof, such as αβ, αγ and βγ may be used. Such receptor isotype specific antagonists may be preferred in order to reduce any side effects associated with the use of non-specific antagonists.

As used herein, “agonist” means a compound that will stimulate the ligand-mediated transactivational activity of the specified retinoid receptor.

As used herein, “antagonist” means a compound that will inhibit or block the ligand-mediated transactivational activity of the specified retinoid receptor.

As used herein, “inverse agonist” means a compound that will decrease a basal level of transactivational activity of the specified retinoid receptor, wherein the basal level is that amount of transactivational activity observed in the absence of added agonist.

As used herein, the term “selective” means that a given ligand demonstrates at least about a 10 fold greater binding affinity, as indicated by, for example, K_{d} value, (dissociation constant) for one receptor subtype than for another receptor subtype.

As used herein, the term “specific” means that a given ligand demonstrates at least about a 500 fold greater binding affinity, and more preferably at least about a 1000 fold greater binding affinity, for one receptor subtype than for another receptor subtype.

As used herein, the term “treating” means reducing or slowing the progression of a disease. Alternatively, or additionally, the term means to remedy or cure a disease. Where the disease is tumor related, the term treating means to inhibit cancer cell growth and/or reduce the sign of a tumor.

As used herein, the term “bone healing” means a pathological condition where cartilage is converted into bone. Alternatively, or additionally, ther term means fracture repair.

As used herein, the term “non-union condition” means a pathological condition where the cartilage conversion to bone is inhibited or blocked.

As used herein, the term “osteoblasts” means the cells which continuously produce bone tissue in adults.

As used herein, the term “osteoclasts” means the cells which destroy bone.

The term “ameliorating” means reducing the symptoms associated with a particular disease, such as pain and inflammation.

In a preferred method of treatment, the antagonist is a compound of formula (I)

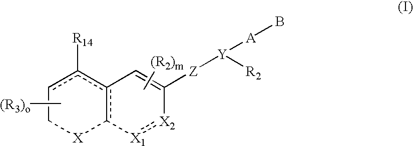

wherein

X is S, SO, SO₂, O, NR₁, [C(R₁)₂]n where each R₁ is independently or together H or alkyl of 1 to 6 carbons, and n is 1 or 2;

or X is absent;

X₁ and X₂ are each C; or

X₁ is absent and X₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons;

provided that at least X is present, or X₁ and X₂ are each C;

— are optionally present bonds;

each R₂ is independently or together hydrogen, lower alkyl of 1 to 6 carbons, F, Cl, Br, I, CF₃, fluoro substituted alkyl of 1 to 6 carbons, OH, SH, alkoxy of 1 to 6 carbons, alkylthio of 1 to 6 carbons, NH₂, NR₁H, N(R₁)₂, N(R₁)COR₁, NR₁CON(R₁)₂ or OCOR₁;

each R₃ is independently or together hydrogen, lower alkyl of 1 to 6 carbons, F, Cl, Br or I;

m is an integer having a value of 0-3;

o is an integer having a value of 0-3;

Z is —C∉C—, —N═N—, —N═CR₁—, —CR₁═N, —(CR₁═CR₁)_{n′}— where n′ is an integer having the value 0-5, —CO—NR₁—, —CS—NR₁—, —NR₁—CO—, —NR₁CS—, —COO—, —OCO—, CSO— or —OCS—;

Y is a phenyl or naphthyl group, or heteroaryl selected from the group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or

when Z is —(CR₁═CR₁)_{n′}— and n′ is 3, 4 or 5 then Y represents a direct valence bond between said —(CR₁═CR₁)_{n′} group and B;

A is (CH₂)_{q} where q is 1-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds; or is a direct bond or is absent;

B is hydrogen, COOH, COOR₈, CONR₉R₁₀, CH₂OH, CH₂OR₁₁, CH₂OCOR₁₁, CHO, CH(OR₁₂)₂, CHOR₁₃O, COR₇, CR₇(OR₁₂)₂, CR₇OR₁₃O, or tri-lower where R₇ is an alkyl, cycloalkyl or alkenyl group containing 1 to 5 carbons, R₈ is an alkyl group of 1 to 10 carbons or (trimethylsilyl)alkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons; and

R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N; r is an integer having a value of 0-6; and

R₁₅ is independently H, F, Cl, Br, I, NO₂, N(R₈)₂, N(R₈)COR₈, NR₈ CON(R₈)₂OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or (trialkylsilyl)oxy group where the alkyl groups independently have 1 to 6 carbons; or

a pharmaceutically acceptable salt or ester thereof.

According to one embodiment, X is present and X₁ is absent, providing compounds of formula (Ia):

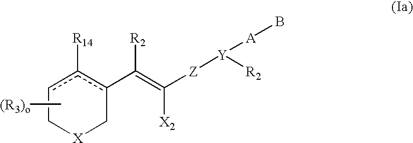

In another embodiment, X is absent and X₁ and X₂ are C, providing compounds of formula (Ib):

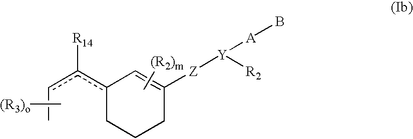

In yet a further particularly preferred embodiment, X is present and X₁ and X₂ are C, providing compounds of formula (Ic):

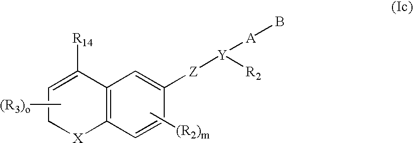

In preferred embodiments of formulas I, Ia, Ib and Ic, Y is phenyl and R₁₄ is (R₁₅)ᵣ-phenyl, where preferably the bond between R₁₄ and the heterocyclic moiety comprising X allows for free rotation of the R₁₄ group. In a further embodiment, —Y(R₂)-A-B is -phenyl-COOH.

Specific antagonists within the scope of formula (I), method of synthesis as well as definitions of terminology used to define compounds of formula (I), are more fully described in U.S. Pat. No. 5,776,699. Further examples of compounds which may be used in practicing the present invention include compounds of formulas (II) through (V):

R₁₄—X′—Y₁(R₂R′₃)—Z—Y(R₂)—A-B (II)

where

X′ is O, S, SO, SO₂, N, NR₃ or C(R₃)₂; or —X′—R₁₄ is —C(R₁₄)H₂ or —C(R₁₄)—(CH₂)ₙH where n is 1-6;

Y₁ is phenyl, naphthyl or heteroaryl selected from the group consisting of pyridyl, thienyl, furyl, pyridazinyl, pyrimidinyl, pyrazinyl, thiazolyl, oxazolyl, imidazolyl and pyrrazolyl, said phenyl, naphthyl and heteroaryl groups being optionally substituted with one R′₃ and one or two R₂ groups;

R′₃ is H, (C₁-C₁₀) alkyl, 1-adamantyl, 2-tetrahydropyranoxy, trialkylsilanyl and trialkylsilanyloxy where alkyl comprises 1 to 6 carbons, alkoxy and alkylthio where alkyl comprises 1 to 10 carbons, or OCH₂O(C₁₋₆)alkyl; and Z, Y, A, B, R₂, R₃ and R₁₄ are as defined above; where preferred embodiments include compounds of formula (IIa):

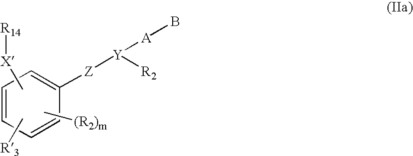

where

m is 0-2; where further preferred embodiments include compounds of formula (IIb):

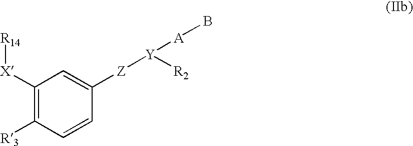

where

preferably R′₃ is alkyl; and where additional embodiments include compounds of formula (IIc):

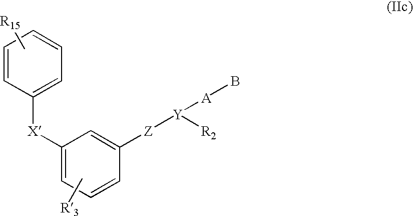

;compounds of formula (III):

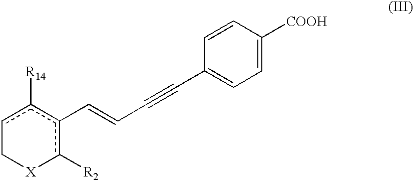

where

R₂ is as described above and additionally preferably C₁-C₆ alkenyl, and X and R₁₄ are as described above;

compounds of formula (IV):

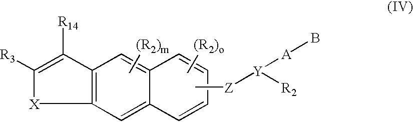

wherein

X is S, SO, SO₂, O, NR₁, [C(R₁)₂]ₙ, —C(R₁)₂—NR₁—, —C(R₁)₂—S—, —C(R₁)₂—O— or —C(R₁)₂—(R₁)₂—, where R₁, R₂, R₃, R₁₄, Z, Y, A, B, m and o are as described above; where preferred embodiments include compounds of formula (IVa):

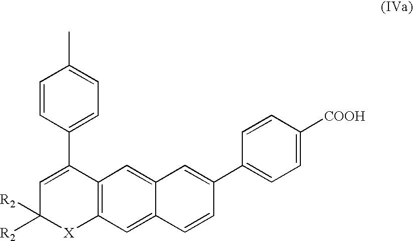

and compounds of formula (V):

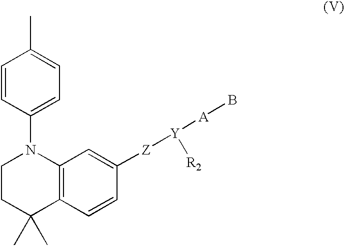

where

Z, Y, A, B and R₂ are as described above.

Another preferred class of compounds are those of formula (VI):

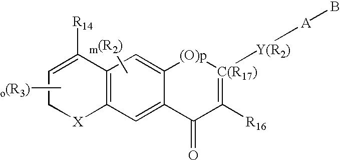

wherein

X, R₂, R₃ m, o, Y, A, B, R₁₄ and R₁₅ are as defined above, and;

R₁₆ is H or lower alkyl of 1 to 6 carbons;

R₁₇ is H, lower alkyl of 1 to 6 carbons, OH or OCOR₁₁, where R₁₁ is defined above, or R₁₇ is absent; and

p is 0 or 1, with the proviso that when p is 1 then R₁₇ is absent.

A further preferred class of compounds are those of formula (VII):

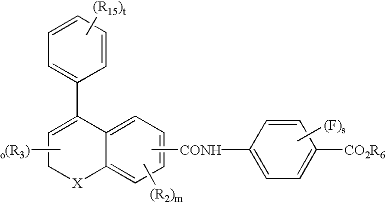

where

X, R₁ R₂ m, R₃ and o are as defined above;

s is an integer having a value of 1-3; and

R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl;

R₁₅ is as defined above;

t is an integer having a value of 0-5, where the CONH group is in the 6 or 7 position of the benzopyran, and in the 2 or 3 position of the dihydronaphthaline ring; or

a pharmaceutically acceptable salt thereof

Another preferred class are compounds of formula (VIII):

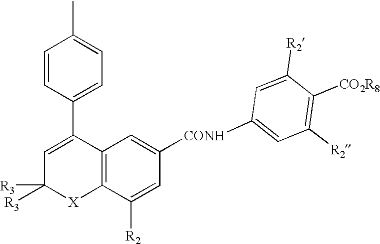

where

X is preferably C(CH₃)₂ or O;

R₂ is preferably H or Br;

R_{2′} and R_{2″} independently are H or F;

R₃ is preferably H or CH₃; and

R₈ is preferably H, lower alkyl of 1 to 6 carbons; or

a pharmaceutically acceptable salt thereof.

A further preferred class of such compounds are of formula (IX):

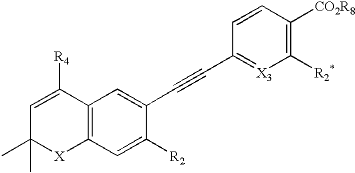

where

X₁ is preferably S or O;

X₃ is CH or N;

R₂ is preferably H, F, CF₃ or alkoxy of 1 to 6 carbons;

R₂* is H, F or CF₃;

R₈ is preferably H, or lower alkyl of 1 to 6 carbons; and

R₁₄ is preferably unsubstituted phenyl, thienyl or pyridyl, or phenyl, thienyl or pyridyl substituted with one to three R₁₅ groups, where R₁₅ is preferably lower alkyl of 1 to 6 carbons, chlorine, CF₃, or alkoxy of 1 to 6 carbons, or

a pharmaceutically acceptable salt thereof.

In a preferred embodiment of compounds of formula (IX), X is S, R₂ is H, F or OCH₃; R₂* is H or F; R₈ is H, or lower alkyl of 1 to 6 carbons; and R₁₄ is selected from the group consisting of phenyl, 4-(lower-alkyl)phenyl, 5-(lower alkyl)-2-thienyl, and 6-(lower alkyl)-3-pyridyl where lower alkyl has 1 to 6 carbons; or a

pharmaceutically acceptable salt thereof. In one particular embodiment, R₂ is H; R₂* is H; X₃ is CH; and R₁₄ is ethyl.

In another preferred embodiment of compounds of formula (IX), X is O; R₂ is H; R₂* is H or F; R₈ is H or lower alkyl of 1 to 6 carbons; and

R₁₄ is selected from the group consisting of phenyl, and 4-(lower-alkyl)phenyl, where lower alkyl has 1 to 6 carbons, or a pharmaceutically acceptable salt thereof.

Yet another preferred group of compounds is of formula (X):

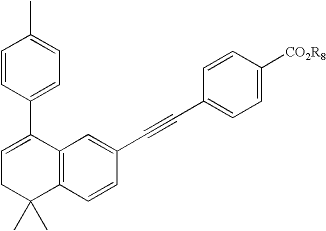

where

R₈ is H, lower alkyl of 1 to 6 carbons, or a pharmaceutically acceptable salt of said compound. When R₈ is H, this compound is AGN 109, a preferred embodiment.

Furthermore, the structures of additional compounds useful in the present invention are disclosed below.

A.

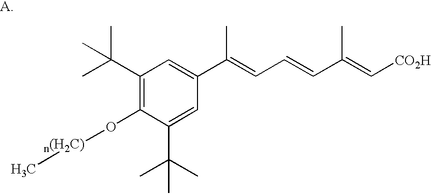

where

n is an integer from 1 to 10.

B.

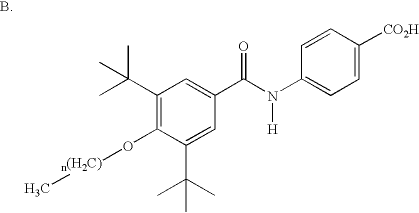

where

n is an integer from 1 to 10.

C.

D.

E.

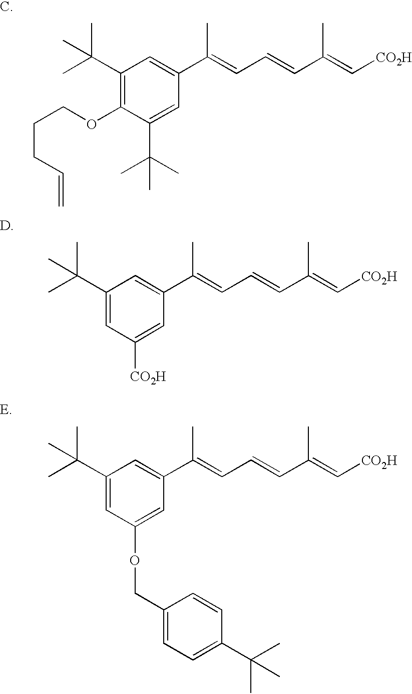

As discussed above, any compound or agent having retinoid receptor antagonist activity may be used. Means for determining antagonist activity of a given agent or compound are known in the art. For example, a holoreceptor transactivation assay and a ligand binding assay which measure the antagonist/agonist like activity of the compounds of the invention, or their ability to bind to the several retinoid receptor subtypes, respectively, are described in published PCT Application No. WO 93/11755 (particularly on pages 30-33 and 37-41) published on Jun. 24, 1993, the specification of which is also incorporated herein by reference.

A pharmaceutically acceptable salt may be prepared for any compound in this invention having a functionality capable of forming a salt, for example, an acid functionality. A pharmaceutically acceptable salt is any salt which retains the activity of the parent compound and does not impart any deleterious or untoward effect on the subject to which it is administered and in the context in which it is administered.

Pharmaceutically acceptable salts may be derived from organic or inorganic bases. The salt may be a mono or polyvalent ion. Of particular interest are the inorganic ions, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Where there is a nitrogen sufficiently basic as to be capable of forming acid addition salts, such may be formed with any inorganic or organic acids or alkylating agent such as methyl iodide. In such cases, preferred salts are those formed with inorganic acids such as hydrochloric acid, sulfuric acid or phosphoric acid. Any of a number of simple organic acids such as mono-, di- or tri-acid may also be used.

Some of the compounds of the present invention may have trans and cis (E and Z) isomers. In addition, the compounds of the present invention may contain one or more chiral centers and therefore may exist in enantiomeric and diastereomeric forms. Still further oxime and related compounds of the present invention may exist in syn and anti isomeric forms. The scope of the present invention is intended to cover all such isomers per se, as well as mixtures of cis and trans isomers, mixtures of syn and anti isomers, mixtures of diastereomers and racemic mixtures of enantiomers (optical isomers) as well. In the present application when no specific mention is made of the configuration (cis, trans, syn or anti or R or S) of a compound (or of an asymmetric carbon) then a mixture of such isomers, or either one of the isomers is intended. In a similar vein, when in the chemical structural formulas of this application a straight line representing a valence bond is drawn to an asymmetric carbon, then isomers of both R and S configuration, as well as their mixtures are intended. Defined stereochemistry about an asymmetric carbon is indicated in the formulas (where applicable) by a solid triangle showing β-configuration, or by a hashed line showing a-configuration.

The present invention also provides pharmaceutical compositions comprising one or more compounds of the invention together with a pharmaceutically acceptable diluent or excipient. Preferably such compositions are in unit dosage forms such as tablets, pills, capsules (including sustained-release or delayed-release formulations), powders, granules, elixirs, tinctures, syrups and emulsions, sterile parenteral solutions or suspensions, aerosol or liquid sprays, drops, ampoules, auto-injector devices or suppositories; for oral, parenteral (e.g., intravenous, intramuscular or subcutaneous), intranasal, sublingual or rectal administration, or for administration by inhalation or insufflation, and may be formulated in an appropriate manner and in accordance with accepted practices such as those disclosed in Remington's Pharmaceutical Sciences, Gennaro, Ed., Mack Publishing Co., Easton Pa., 1990. Alternatively, the compositions may be in sustained-release form suitable, for example, for once-weekly or once-monthly administration; for example, an insoluble salt of the active compound, such as the decanoate salt, may be adapted to provide a depot preparation for intramuscular injection. The present invention also contemplates providing suitable topical formulations for administration to, e.g. eye or skin or mucosa.

For instance, for oral administration in the form of a tablet or capsule, the active drug component can be combined with an oral, non-toxic pharmaceutically acceptable inert carrier such as ethanol, pharmaceutically acceptable oils, glycerol, water and the like. Moreover, when desired or necessary, suitable binders, lubricants, disintegrating agents, flavoring agents and coloring agents can also be incorporated into the mixture. Suitable binders include, without limitation, starch, gelatin, natural sugars such as glucose or beta-lactose, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For preparing solid compositions such as tablets, the active ingredient may be mixed with a suitable pharmaceutical excipient, e.g., such as the ones described above, and other pharmaceutical diluents, e.g., water, to form a solid preformulation composition containing a homogeneous mixture of a compound of the present invention, or a pharmaceutically acceptable salt thereof. By the term “homogeneous” is meant that the active ingredient is dispersed evenly throughout the composition so that the composition may be readily subdivided into equally effective unit dosage forms such as tablets, pills and capsules. The solid preformulation composition may then be subdivided into unit dosage forms of the type described above containing from 0.1 to about 50 mg of the active ingredient of the present invention.

In another embodiment, the tablets or pills of the present composition may be coated or otherwise compounded to provide a dosage form affording the advantage of prolonged action. For example, the tablet or pill can comprise an inner core containing the active compound and an outer layer as a coating surrounding the core. The outer coating may be an enteric layer which serves to resist disintegration in the stomach and permits the inner core to pass intact into the duodenum or to be delayed in release. A variety of materials can be used for such enteric layers or coatings, such materials including a number of polymeric acids and mixtures of polymeric acids with conventional materials such as shellac, cetyl alcohol and cellulose acetate.

The liquid forms in which the present compositions may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical carriers. Suitable dispersing or suspending agents for aqueous suspensions include synthetic and natural gums such as tragacanth, acacia, alginate, dextran, sodium carboxymethylcellulose, gelatin, methylcellulose or polyvinyl-pyrrolidone. Other dispersing agents which may be employed include glycerin and the like. For parenteral administration, sterile suspensions and solutions are desired. Isotonic preparations which generally contain suitable preservatives are employed when intravenous administration is desired. The compositions can also be formulated as an ophthalmic solution or suspension formation, i.e., eye drops, for ocular administration.

The term “subject,” as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

Advantageously, compounds of the present invention may be administered in a single daily dose, or the total daily dosage may be administered in divided doses two, three or four times daily. Furthermore, compounds for the present invention may be administered in intranasal form via topical use of suitable intranasal vehicles, or via transdermal routes, using those forms of transdermal skin patches well known to persons skilled in the art. To be administered in the form of a transdermal delivery system, the dosage administration will, of course, be continuous rather than intermittent throughout the dosage regimen, and dosage levels will require that this be taken into consideration when formulated.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound employed. A physician or veterinarian of ordinary skill can readily determine and prescribe the effective amount of the drug required to prevent, counter or arrest the progress of the disease or disorder which is being treated.

The daily dosage of retinoid receptor antagonists or reverse agonists may vary over a wide range from 0.01 to 100 mg per adult human per day. For oral administration, the compositions are preferably provided in the form of tablets containing 0.01, 0.05, 0.1, 0.5, 1.0, 2.5, 5.0, 10.0, 15.0, 25.0 or 50.0 mg of the active ingredient for the symptomatic adjustment of the dosage to the patient to be treated. A unit dose typically contains from about 0.001 mg to about 50 mg of the active ingredient, preferably from about 1 mg to about 10 mg of active ingredient. An effective amount of the drug is ordinarily supplied at a dosage level of from about 0.0001 mg/kg to about 25 mg/kg of body weight per day. Preferably, the range is from about 0.001 to 10 mg/kg of body weight per day, and especially from about 0.001 mg/kg to 1 mg/kg of body weight per day. The compounds may be administered on a regimen of 1 to 4 times per day.

In another embodiment the instant invention is drawn to the use of retinoid receptor agonists as positive regulators of endochondral ossification. In this embodiment are provided methods for (a)enhancing the reparative process during fracture repair, (b) treating congenital conditions in individuals who may exhibit poor or retarded growth and ossification, (c)ameliorating osteoporosis, and (d) stimulating and modulating intramembrane ossification through treatment with retinoid receptor agonists. Congenital conditions of poor and retarded ossification may included, by way of example only and not of limitation, spondyloepiphyseal dysplasia congenita, skeletal dysplasias, hip dysplasia, and multiple epiphyseal dysplasias.

The synthesis and structures of exemplary retinoid receptor agonists is described, by way of example only and not of limitation, in U.S. Pat. Nos. 5,808,124; 5,763,635; 5,747,542; 5,741,896; 5,723,666; 5,688,957; 5,618,943; 5,618,931; 5,616,712; 5,556,996; 5,543,534; 5,534,641; 5,514,825; 5,498,795; 5,498,755; 5,489,584; 5,475,022; 5,470,999; 5,451,605; 5,426,118; 5,399,561; 5,391,753; 5,346,915; 5,346,895; 5,344,959; 5,326,898; 5,134,159; 5,945,551; 5,015,658; 5,013,744; 5,006,550; 4,992,468; and 4,980,369, all of which are incorporated herein by reference in their entireties.

It is within the applicant's contemplation that any retinoid receptor agonist presently known in the art, or subsequently developed, may be used in practicing the claimed methods.

All references cited are incorporated herein by reference in their entireties.

The invention is disclosed in further detail in the following examples, which are not in any way intended to limit the scope of the invention as claimed.

### EXAMPLES

### (a) Example I

Materials and Methods

(b) In situ Hybridization

This procedure was carried out as described previously (Noji et al., Acta Histochem. Cytochem. 23, 353-366 (1990); Koyama et al., Dev. Dynam. 203, 152-162 (1995)). Briefly, chick embryos or embryo parts were fixed with 4% paraformaldehyde for 4 hr or overnight, embedded in paraffin and sectioned. The 5 μm thick sections were pretreated with 1 μg/ml proteinase K (Sigma, St. Louis, Mo.) in 50 mM Tris, 5 mM EDTA, pH 7.5 at room temperature for 1 min, immediately postfixed in 4% paraformaldehyde buffer for 10 min, and then washed twice in PBS containing 2 mg/ml glycine for 10 min/wash. Sections were treated for 15 min with a freshly prepared solution of 0.25% acetic anhydride in triethanolamine buffer. Sections were hybridized with ³⁵S-labeled antisense or sense chick cDNA riboprobes (approximately 1×10⁶ DPM/section) at 50° C. for 16 hr. After hybridization, slides were washed three times with 2×SSC containing 50% formamide at 50° C. for 20 min/wash, treated with 20 μg/ml RNaseA for 30 min at 37° C., and finally washed three times with 0.1×SSC at 50° C. for 10 min/wash. Sections were coated with Kodak NTB3 emulsion diluted 1:1 with water, exposed for 7 days, and developed with Kodak D19 for 3 min at 20° C. After staining with hematoxylin and eosin, slides were analyzed with a Nikon microscope using bright and dark field optics.

The chick cDNA probes used were: the 1.6 kb RARa and 0.9 kb RARβ clones encompassing the ligand binding domain (Noji et al., Nature 350, 83-86 (1991)); a 0.16 kb RARγ subclone (nucleotides 444-607) prepared from full length RARγ2 (Michaille et al., Dev. Dynam. 201, 334-343 (1994)) and encoding a portion of domain C; a 0.56 kb Ihh clone encoding part of the N-terminal domain (Vortkamp et al., Science 273, 613-633 (1996)); the type I collagen pGEM821, a 0.821 kb clone from the 3′ end of type I collagen subunit a2(I) (Bennett et al., J. Biol. Chem. 264, 8402-8409 (1989)); the type II collagen clone pDLr2 (Leboy et al., J. Biol. Chem. 264, 17281-17286 (1989)), a 0.8 kb clone from the 3′ region of type II collagen (Young et al., Nucl. Acids Res. 12, 4207-4228 (1984)); the 0.197 kb type X collagen clone pDLr10 (Leboy et al., J. Biol. Chem. 264, 17281-17286 (1989)); and the 1.1 kb clone pMMPP2 containing the full coding sequence of osteopontin (Moore et al., Biochemistry 30, 2501-2508 (1991)).

Antagonist Treatment

The RAR antagonists used were AGN 109 (Allergan Pharmaceuticals, Irvine, Calif.) and Ro 41-5253 (shown below) (Hoffmann-LaRoche, Basel, Switzerland).

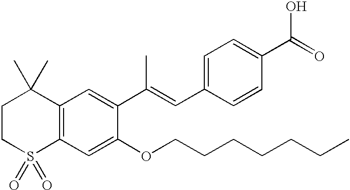

Ro 41-5253 exerts antagonistic effects on all RAR isoforms but preferentially on RARa (IC₅₀=60 nM); its IC₅₀ for RARγ is 3300 nM (Apfel et al., Proc. Natl. Acad. Sci. USA 89, 7129-7133 (1992); Keidel et al., Mol. Cell. Biol. 14, 287-298 (1994)). AGN 109 inhibits equally well RARa, β and γ, and has a nearly 500-fold lower IC₅₀ for RARγ (5+1 nM) (Klein et al., J. Biol. Chem. 271, 22692-22696 (1996)) compared to Ro 41-5253. AG1-X2 ion-exchange beads of 200-400 μm in diameter were soaked for 1 hr in solutions of Ro 41-5253 or AGN 109 at concentrations ranging from 3.5 μM to 3.5 mM. This range of concentrations was based on previous studies (see, for example, Lu et al., Development 124, 1643-1651 (1997)). Antagonist solutions were prepared in DMSO and used under yellow light conditions; control beads were soaked in DMSO alone. Beads were then dipped very briefly in phenol red-containing saline (HBSS) so that they were more readily visible during implantation.

Antagonist-containing or control beads were implanted in the wing bud of stage 21-22 (Day 3-3.5) or stage 27-28 (Day 5.5) chick embryos (Hamburger and Hamilton, J. Morphol. 88, 49-92 (1951)); contralateral wing bud served as control. A small window was opened in the egg shell and a small incision was made on the antero-dorsal proximal portion of the bud. One bead or several beads were then placed in the vicinity of the prospective humerus as specified below, and eggs were sealed and returned to the incubator. On the day of analysis, embryos were sacrificed by decapitation, and control and operated wings were examined by microscopy, using a Nikon SMZ-U dissecting photomicroscope, and humerus length was measured micrometrically. Because length of control humeri varied slightly from embryo to embryo, possibly reflecting slight differences in age, humeri were considered affected by antagonist treatment only if their length was shortened at least 25% over control value. Companion control and antagonist-treated limbs were processed for histology and in situ hybridization using tissue sections.

Chondrocyte Cultures

Cell populations rich in prehypertrophic and early hypertrophic chondrocytes were isolated from the cephalic core region of Day 17-18 chick embryo sterna, while immature chondrocytes were isolated from the caudal sternal region (Gibson and Flint, J. Cell Biol. 101, 277-284 (1985); Pacifici et al., Exp. Cell Res. 195, 38-46 (1991); Iwamoto et al., Exp. Cell Res. 207, 413-420 (1993b)). The dissected cephalic and caudal tissues were incubated for 1 hr at 37° C. in saline containing 0.1% type 1-S collagenase (Sigma Chemical Co., St. Louis, Mo.); the cells released after this incubation were discarded as they consisted mainly of perichondrial and blood cells. The remaining tissue was incubated in a fresh mixture of 0.25% trypsin and 0.1% collagenase for 3 hr at which point it was completely digested. The freshly isolated chondrocytes were plated at a density of 2×10⁵ cells/well in 12-well plates, 1×10⁶ cells/60 mm dish or 3×10⁶ cells/100 mm dish. The cephalic core chondrocytes were grown continuously, without subculturing, for 2 to 3 weeks in monolayer. During the first 2 days, cultures received 4 U/ml of testicular hyaluronidase to minimize cell detachment (Leboy et al., J. Biol. Chem. 264, 17281-17286 (1989)), and cultures became confluent by 2 weeks. The caudal immature chondrocytes were first grown for 5 days at which point floating immature chondrocytes were separated from attached contaminating fibroblastic cells. The floating cells were trypsinized and replated in secondary cultures in the presence of hyaluronidase to increase cell attachment. Cultures were fed every other day with Dulbecco's modified high-glucose Eagle's medium (GIBCO BRL, Gaithersburg, Md.) containing 10% defined fetal calf serum (Hyclone, Logan, Utah), 2 mM L-glutamine, and 50 U/ml penicillin and streptomycin (Pacifici et al., Exp. Cell Res. 195, 38-46 (1991)). When indicated, cultures were treated with all-trans-RA (Sigma) or combinations of all-trans-RA and Ro 41-5253. Stock solutions of these retinoids were prepared in DMSO and were diluted into working solutions in 95% ethanol; control dishes received an equivalent amount of vehicle without retinoids. To analyze mineralization, cephalic sternal control and retinoid-treated cultures were supplemented with 3 mM β-glycerophosphate to serve as a phosphate source. During these various regimens, medium was changed daily. To localize calcium deposits, the cell layers were stained with 0.5% alizarin red S solution, pH 4.0, for 5 min at room temperature. In experiments in which cultures were treated for 2, 4 or 6 days, retinoid treatments were initiated so that all cultures (including control cultures) were harvested simultaneously.

RNA Isolation and Analysis

Whole cellular RNA isolated from chick embryo cartilages and cultured chondrocytes by the guanidine isothiocynate method (Chomczynski and Sacchi, Anal. Biochem. 162, 156-159 (1987)) was denatured by glyoxalation, electrophoresed on 1% agarose gels at 10 or 30 μ/lane, and transferred to Hybond-N membranes by capillary blotting, as described previously (Oettinger and Pacifici, Exp. Cell Res. 191, 292-298 (1990); Iwamoto et al., Exp. Cell Res. 205, 213-224 (1993 a)). Blots were stained with 0.04% methylene blue to verify that each sample had been transferred efficiently. Blots were hybridized for 16 hr to ³²P-labeled riboprobes at a concentration of 2.5×10⁶ DPM/ml of hybridization solution containing 50% formamide, 1.5×SSPE, 500 μg/ml sheared denatured salmon sperm DNA, 100 μg/ml tRNA, 0.5% (w/v) dry milk, and 1% SDS. The cDNA probes used were the same as those used for in situ hybridization. Hybridization temperature was 55° C. for RARγ and APase, and 60° C. for type X collagen. After hybridization, blots were rinsed several times at room temperature with 2×SSC and 0.5% SDS; a final high stringency rinse was with 0.1×SSC and 0.5% SDS at 70° C. Blots were exposed to Kodak BioMax x-ray films at −70° C.

Retinoid Analysis

Semi-quantitative analysis of endogenous retinoid levels in embryonic tissues was carried out using a sensitive in vitro reporter assay (Wagner et al., Development 116, 55-66 (1992); McCaffery et al., Development 115, 371-382 (1992)). The β-gal assay consists of an F9 teratocarcinoma cell line stably transfected with a reporter construct which contains a 64 bp retinoic acid-response element (RARE) from the promoter region of the human RARβ gene (Ellis et al., Nature 343, 377-381 (1990)) placed immediately upstream of the E. coli lacZ gene. The F9 cell line constitutively expresses RARα, β and γ (Zelent et al., Nature 339, 714-717 (1989)), which confer retinoid responsivity to the stably transfected construct. Cells were maintained on gelatin-coated dishes in modified L15 CO₂ tissue culture medium (Specialty Media, Lavallette, N.J.) supplemented with 20% fetal calf serum and 0.8 mg/ml G418 (complete medium), and were used when 80-90% confluent. In this culture condition, the reporter cells have been shown to be very sensitive (i.e., high expression of β-gal) to exogenous all-trans-RA treatment at concentrations as low as 0.01 nM (Wagner et al., Development 116, 55-66 (1992)). In these cells exogenous 9-cis-RA, a ligand for both RXRs and RARs (Levin et al., Nature 355, 359-361 (1992)), stimulates transcription with a 10-fold lower efficiency than in response to all-trans-RA treatment (unpublished observations).

To prepare tissue extracts, tissues were surgically isolated from Day 10 chick embryos and included. The metaphyseal-diaphyseal portion of cartilaginous humerus and tibia from which adherent perichondral tissues were carefully removed, liver, brain, gizzard and heart. During isolation, all tissues were kept in saline on ice under yellow safety light conditions to protect the retinoids. About 200 mg of each tissue or organ were then homogenized with a Polytron in 0.9 ml of L15 complete medium at 4° C. and samples were then quick-frozen in dry ice for complete cell disruption. Samples were thawed in iced water and were incubated at 4° C. for 1 hr to extract retinoids. Extracts were centrifuged at 13,000 g for 15 min at 4° C. The resulting supernatants were carefully separated from the pellet and directly added to semiconfluent cultures of F9 reporter cells grown in 22 mm multiwell plates (0.4 ml/well). Cultures were reincubated for 24 hr and were then processed for histochemical detection of β-galactosidase activity (Lim and Chae, Biotechniques 7, 576, 579 (1989)).

To confirm that β-galactosidase activity was proportional to retinoid concentration, parallel cultures of semiconfluent F9 cell cultures were treated with known amounts of all-trans-RA ranging from 1 M to 2 μM (from 100×stock solutions in 95% ethanol), incubated for 24 hrs and then processed for quantitative analysis of β-galactosidase activity. Briefly, cultures were fixed with 0.1% glutaraldehyde in 0.1 M phosphate buffer pH 7.0 for 15 min at room temperature. After rinsing with PBS, cultures were stained with a solution of 0.2% X-Gal in phosphate buffer for 16 hrs at 37° C. After rinsing again, cultures were extracted with 0.2 ml of DMSO and absorbance of the extracted material was determined at 655 nm using a Perkin-Elmer spectrophotometer. Under these conditions, the F9 cells exhibited a linear increase in β-galactosidase activity between 1 nM to 0.5 μM all-trans-RA.

### Example II

Results

RAR Gene Expression During Skeletogenesis

In a first set of experiments (see Example I, In situ Hybridization), the expression patterns of RARα, β and γ were determined at different stages of chick limb skeletogenesis. Longitudinal serial sections of limb skeletal elements were processed for in situ hybridization using ³⁵S-labeled antisense riboprobes encoding antisense chick RARα, β or γ; as controls, sections were hybridized with radiolabeled sense probes targeted to these RAR's. When early newly-emerged skeletal elements were examined, such as the stage 27-28 (Day 5.5) chick embryo humerus which contains only immature chondrocytes and does not yet display growth plates, it was found that the gene expression levels of RARα and γ were low and diffuse, the level of hybridization signal within the newly-formed cartilaginous tissue was somewhat lower than that detectable in the surrounding mesenchymal and connective tissues. In contrast to the diffuse nondescript patterns of RARα and γ, gene expression of RARβ was distinct and quite pronounced in the perichondrial tissue, particularly along the incipient diaphysis, though it was very low in the cartilaginous tissue itself. Hybridization with sense RAR probes yielded barely detectable signal. The overall cartilaginous tissue was delineated by hybridization with a type II collagen antisense probe.

Between Days 8 and 10 of limb development, the long bone cartilaginous models acquire more definitive morphological characteristics and organization. They displayed prospective articular chondrocytes (ac) at their epiphyseal ends and long growth plates with well defined proliferative (pz), postproliferative-prehypertrophic (phz) and hypertrophic (hz) zones occupying the metaphysis and diaphysis. In addition, the diaphysis begins the process of endochondral ossification and is surrounded by an intramembranous bony collar (Fell, J. Morphol. Physiol. 40, 417-459 (1925); Scott-Savage and Hall, J. Morphol 162, 453-464 (1979); Osdoby and Caplan, Dev. Biol. 86, 147-156 (1981); Koyama et al., Dev. Dynam. 203, 152-162 (1995)). In situ hybridization on serial sections of Day 10 chick embryo wing showed that while RARa gene expression remained low and diffuse throughout the cartilaginous tissue and RARβ expression was still strong in perichondrium, RARγ expression was markedly up-regulated in the hypertrophic zone of growth plate. Hybridization with a probe encoding type X collagen, a marker of hypertrophic chondrocytes (Gibson and Flint, J. Cell Biol. 101, 277-284 (1985)), confirmed that there was a significant similarity between the topographical distribution of type X collagen transcripts and RARγ transcripts, though the increase in RARγ transcripts slightly preceded that in type X collagen transcripts. Analysis of other markers revealed that the RARγ- and type X collagen-rich chondrocytes were preceded in the growth plate by prehypertrophic chondrocytes expressing the morphogenetic factor Indian hedgehog (Ihh) (Koyama et al., Dev. Dynam. 207, 344-354 (1996a); Vortkamp et al., Science 273, 613-622 (1996)), and were followed by mineralizing post-hypertrophic chondrocytes undergoing endochondral ossification and expressing late maturation markers such as osteopontin (Iwamoto et al., Exp. Cell Res. 207, 413-420 (1993b)). Osteopontin expression was also detectable in the developing bony collar surrounding the diaphysis and metaphysis. As expected, type II collagen gene expression was strong throughout most of the cartilaginous tissue but was markedly down-regulated in the mineralizing and endochondral ossification zones, while type I collagen RNA was confined to the bony collar, perichondrial tissue and other surrounding connective tissues. Similar results were obtained with Day 8.5 (stage 35) embryos (see below).

The relationship between increased RARβ expression and emergence of hypertrophic chondrocytes was further analyzed in the digit area of Day 10 limbs, which contains short skeletal elements at different stages of development along the proximal-to-dital axis in close proximity to each other. Indeed, it was found that the developmentally older proximal phalangeal (pp) elements contained abundant RARγ transcripts and numerous hypertrophic chondrocytes in the diaphysis, whereas the developmentally younger medial phalange (mp) contained fewer hypertrophic cells and lower amounts of RARγ transcripts and the even younger distal phalange (dp) contained neither. Closer inspection of the diaphyseal region of the proximal phalange revealed that whereas the RARγ transcripts were present throughout the diaphysis, the hypertrophic chondrocytes were not. These cells were much more obvious and numerous at the periphery of the diaphysis than its center.

Taken together, the above data indicate that the RARs display differential patterns of gene expression during limb chondrocyte maturation and skeletogenesis. In particular, while RARα expression remains broad and diffuse, RARγ expression is selectively up-regulated just before the chondrocytes become fully hypertrophic and remains high in the hypertrophic cells. The data also indicate that the first hypertrophic chondrocytes form at the periphery of cartilaginous elements.

Retinoid Bioassays

It was determined next whether the cartilaginous skeletal elements present in limbs at later stages of development also contain endogenous retinoids (see Example I, Retinoid Analysis). If so, the retinoids could serve as ligands for the RARs expressed at those stages. In addition, they could have a direct or indirect role in regulating RAR gene expression itself. As an approach, a sensitive bioassay was used that has been previously used to estimate endogenous retinoid levels in other developing tissues and organs in chick and mouse embryos (Wagner et al., Development 116, 55-66 (1992); McCaffery et al., Development 115, 371-382 (1992)). This bioassay utilizes an F9 teratocarcinoma cell line stably transfected with a retinoid-sensitive RARE/β-galactosidase reporter construct.

The entire cartilaginous humerus was microsurgically isolated from Day 5.5 (stage 27-28) embryos and the metaphyseal-diaphyseal portion of humerus from Days 8.5 and 10 chick embryos. The cartilaginous tissue was then carefully separated from the surrounding perichondrial tissues and the cartilaginous tissue processed for retinoid analysis. For comparison, the perichondrial tissues themselves were processed for analysis as well as liver, brain, eye and skin from the same Day 5.5, 8.5 and 10 embryos. Perichondrial tissues from Day 5.5 embryos, however, were excluded from analysis because they could not be obtained in sufficient quantities given the small size of the embryos. One hundred to 200 mg of each tissue or organ were suspended in fresh complete culture medium, homogenized and extracted; after clarification, the extracts were added to semiconfluent cultures of reporter F9 cells grown in 12 well plates. Cultures were reincubated for 24 hr and were then processed for histochemical detection of β-galactosidase activity. Negative control wells received mock-extracted fresh complete medium; positive control wells received fresh medium containing known amounts of all-trans-RA.

It was found that the cartilaginous tissues contained agents capable of stimulating transcription of the RAR reporter gene and did so at each stage of development examined. The amounts of retinoids in cartilage tissue extracts were much lower than those in liver, eye and skin as to be expected on the basis of the large quantities of retinoids present in these organs, but were higher than those present in brain extracts. Strikingly, it was also found that perichondrial tissues displayed extremely large amounts of retinoids. Negative and positive controls gave predictable results; F9 cells receiving vehicle alone (95% ethanol) were negative, while cells treated with 3 nM all-trans-RA were positive.

Retinoid Antagonists Derange Skeletal Development in vivo

Having shown that RAR gene expression changes during chondrocyte maturation and that the cartilaginous elements as well as their surrounding perichondrial tissues contain endogenous retinoids, experiments were carried out to determine what roles the RARs and their ligands may play during chondrocyte maturation and skeletogenesis (see Example I, Antagonist Treatment). To approach this question, a bead containing retinoid antagonists was implanted in the vicinity of the prospective humeral mesenchymal condensation in stage 21-22 (Day 3-3.5) chick embryos and determined whether humerus development had been impaired by Day 10 in vivo. A bead containing Ro 41-5253 or AGN 109 at concentrations ranging from 3.5 μM to 3.5 mM was placed in one wing bud; the contralateral wing bud received a bead containing vehicle alone and served as control.

Both antagonists had striking effects on humerus development. The humerus of Day 10 embryos implanted with a Ro 41-5253-containing bead was about 50% shorter than control contralateral humerus treated with vehicle alone or untreated humerus. The effects were highly selective and topographically limited to the humerus; no obvious changes in size and/or shape were observed in the developing radius, ulna and digits. Similar effects were exerted by AGN 109, but much lower concentrations of this antagonist were required to obtain high frequency of humeral defects, possibly because of its ability to antagonize every RAR equally well (See Table I).

TABLE I
Dose-dependent effects of retinoid antagonists
on humerus development

| Chick embryo | Treatment/ | | % Normal | % Limbs with |
|---|---|---|---|---|
| Days | Dose | n* | limbs | shortened humerus** |
| 21-22 | none | 7 | 100 | 0 (0/7) |
| 21-22 | Ro 3.5 μM | 8 | 75 | 25 (2/8) |
| 21-22 | Ro 3.5 μM | 9 | 33 | 67 (6/9) |
| 21-22 | AGN 3.5 μM | 10 | 60 | 40 (4/10) |
| 21-22 | AGN 3.5 μM | 6 | 0 | 100 |

*Total number of embryos used. Note that control embryos (indicated as “none”) were implanted with a control bead filled with vehicle alone.
**Humerus was considered affected if it was at least 25% shorter than control.
***Two of these embryos had a shortened ulna or radius also.

Histological and in situ hybridization analyses of longitudinal sections of Day 10 humeri provided further details of the effects of the antagonists. In control humeri the epiphyses and metaphyses were well developed, and the diaphysis contained numerous maturing hypertrophic chondrocytes expressing RARγ and type X collagen, displayed a central core region undergoing replacement by bone and marrow and strongly expressing osteopontin and was surrounded by a thin intramembranous bony collar also expressing osteopontin.

In sharp contrast, the diaphysis of antagonist-treated humeri contained only small-sized chondrocytes expressing neither RARγ nor osteopontin and type X collagen, was completely cartilaginous, and had not undergone endochondral ossification nor marrow invasion. Interestingly, however, the diaphysis was surrounded by a seemingly normal intramembranous bone collar that expressed osteopontin, and the metaphyseal portions displayed Ihh gene expression as seen in control. It is also interesting to note that antagonist-treated humeri often displayed a curvature, with the concave side facing the antagonist-filled bead and the convex side facing the opposite side. No such curvature was observed in control humeri implanted with vehicle-filled bead. The effects elicited by the antagonists were limited to the humerus while skeletal elements distant from the site of bead implantation were normal in both morphology and gene expression, as exemplified by strong type X collagen gene expression in the ulnae of control and antagonist-implanted wings This reiterated the conclusion above that the inhibitory effects exerted by the retinoid antagonists were limited to the site of bead implantation and did not reflect generalized systemic effects.

In the next set of experiments, the issue was addressed whether antagonist treatment initiated at later stages of development would still lead to inhibition of humerus development. If so, this would correlate well with bioassay data showing that endogenous retinoids are continuously present in the cartilaginous tissues and suggesting that retinoids may be continuously required for skeletal development. The treatment period was also shortened as to minimize the interval between experimental manipulation and analysis of the effects. Thus, a single or multiple AGN 109-filled beads were implanted on one side or around the cartilaginous humerus in Day 5.5 (stage 28) chick embryos and the effects examined on Day 8.5. It was found that humerus development had been inhibited even after such short treatment timeframe when implanted with 3-4 beads (6/7); a single bead was not very effective (5/5). Compared to their normal counterparts, the antagonist-treated humeri were shorter, and their cells had not advanced to the hypertrophic stage and lacked transcripts encoding RARγ and type X collagen. Both control and treated humeri exhibited very strong expression of type II collagen, indicating that the antagonist was not exerting unwanted side effects on cell viability and differentiated functions.

These experiments produced two additional interesting data. The first one was that in control Day 8.5 humerus the first type X collagen-expressing chondrocytes emerged at the periphery of the diaphysis. This data is in perfect agreement with morphological observations above and was confirmed by in situ hybridization on serial sections throughout the diaphysis. The second interesting data was that the antagonist-treated humeri were morphologically straight as the controls and never displayed a curvature, possibly because the antagonist-filled beads had been placed on both sides of the humeri.

To determine whether the effects of antagonist treatment were reversible and would dissipate with time and further development, embryos implanted with AGN 109 beads at stage 28 (Day 5.5) as above were allowed to develop until Days 14 to 18 of embryogenesis and were then processed for histology and in situ hybridization. It was found that by Day 14 the antagonist-treated humeri contained hypertrophic chondrocytes in their diaphysis exhibiting characteristic gene expression patterns, that is strong type X collagen and low type II collagen gene expression. In addition, bone and bone marrow progenitor cells had begun to invade the hypertrophic cartilage. These morphological and gene expression features normally characterize the humerus around Day 9-9.5 of embryogenesis, indicating that development of antagonist-treated humerus had been delayed by about 5 days but was now resuming its normal course.

Cultured Chondrocytes

In a final set of studies, it was determined whether the antagonists used in the above in vivo experiments are able to antagonize the biological effects of natural retinoids in chondrocytes and whether the antagonists were able to block or inhibit the pro-maturation effects of exogenous all-trans-RA on cultures of chick embryo chondrocytes (see Example I, Chondroycte Cultures). As shown previously, cultures of immature chondrocytes isolated from the caudal resting portion of Day 17-18 chick embryo sternum require treatment with all-trans-RA to develop into hypertrophic type X collagen-expressing cells. Likewise, cultures of newly-emerged hyper-trophic chondrocytes isolated from the cephalic portion of Day 17-18 chick embryo sternum require all-trans-RA treatment to complete their maturation into post-hypertrophic alkaline phosphatase-rich, mineralizing chondrocytes (Pacifici et al., Exp. Cell Res. 195, 38-46 (1991); Iwamoto et al., Exp. Cell Res. 207, 413-420 (1993b); Microsc. Res. Tech. 28, 483-491 (1994)).

Thus, immature caudal sternal chondrocytes were grown in standard serum containing cultures for about 2 weeks. During this period, the cells actively proliferated and increased moderately in size (about 2-3 fold), indicating that they had advanced to a pre-hypertrophic stage of maturation (see Pacifici et al., Exp. Cell Res. 195, 38-46 (1991)). Cultures were then treated with all-trans-RA, Ro 41-5253, both all-trans-RA and Ro 41-5253, or left untreated. Northern blot analysis showed that control untreated cultures contained barely detectable amounts of type X collagen transcripts;. However, cultures treated for 2, 4 or 6 days with 50 nM all-trans-RA displayed a marked time-dependent increase in type X collagen transcripts. Such increase was significantly, though not totally, blocked by co-treatment with 500 nM Ro 41-5253. Treatment with antagonist alone did not have major effects. Thus, Ro 41-5253 is able to counteract the up-regulation of an early maturation marker, type X collagen, in cultured pre-hypertrophic caudal sternal chondrocytes.

This conclusion was confirmed and extended with cultures of more mature chondrocytes isolated from the cephalic core portion of sternum. Two week-old control untreated cultures displayed the expected hypertrophic cell phenotype characterized by a large cell diameter (see Pacifici et al., Exp. Cell Res. 195, 38-46 (1991)) and abundant type X collagen mRNA. When the cells were treated with 50 nM all-trans-RA, gene expression of the late maturation marker alkaline phosphatase was increased dramatically, while expression of type X collagen was essentially eliminated by 6 days of treatment, in excellent correlation with the fact that alkaline phosphatase expression is up-regulated and type X collagen expression is down-regulated in vivo when hypertrophic chondrocytes advance to their terminal post-hypertrophic mineralizing stage during endochondral ossification (Iwamoto et al., Micros. Res. Tech. 28, 483-491 (1994)). The opposite responses of these two genes to all-trans-RA treatment were counteracted by co-treatment with 500 nM Ro 41-5253. Thus, alkaline phosphatase gene expression remained quite low while type X collagen gene expression remained fairly strong. Treatment with antagonist alone had no major effects. Similar data were obtained with AGN 109.

To examine the mineralizing stage of the chondrocyte maturation process, maturing chondrocytes isolated from the cephalic core portion of sternum were grown for 2 weeks in 22 mm multiwell plates until confluent and were then treated for 6 days with all-trans-RA, both all-trans-RA and Ro 41-5253, or Ro 41-5253 alone. All cultures received β-glycerophosphate, a phosphate donor needed for mineral formation and deposition; mineral was revealed by staining with alizarin red. Control untreated cultures exhibited no detectable staining. In contrast, cultures treated with 25 or 50 nM all-trans-RA contained abundant alizarin red-stainable mineral. Increasing amounts of Ro 41-5253 did effectively antagonize the pro-mineralization effects of all-trans-RA such that cultures co-treated with 25 or 50 nM all-trans-RA and 500 nM Ro 41-5253 exhibited almost no mineralization. Treatment with Ro 41-5253 alone had no effects.

Thus, exogenous all-trans-RA induces changes in gene expression, cell behavior and activities in cultured sternal chondrocytes which are identical to those occurring at the different stages of chondrocyte maturation in vivo. The retinoid antagonists used counteract the pro-maturation abilities of all-trans-RA.

### Example III

Bone Healing

A subject having a simple fracture of the humorus suffers from a congenital condition giving rise to poor natural bone healing. The patient's medical history shows that bone healing has typically taken 2-4 times longer in this patient than is seen in patients lacking such a condition.

Treatment is delayed for a period of time to permit infiltration of cartilage-forming cells into the space between the broken parts of the humorus. Bone healing is monitored daily by x-ray during the treatment regimen. Within 4 days of treatment normal matrix formation is seen, and the patient then treated by oral administration of a therapeutic amount of an RAR receptor agonist. The effective dosage is chosen in accordance with factors including the activity of the drug, and the age, weight, sex and medical condition of the patient and the oral route of administration. X-ray monitoring is continued during the course of therapy.

Monitoring during administration of the RAR agonist reveals that ossification occurs at a rate within the normal time course of bone healing for fractures of this type, particularly in comparison to the patient's prior medical history. The patient is able to resume use of the affected arm within a time considered normal.

### Example IV

Osteoporosis

A woman, 61 years old, suffers osteoporosis characterized by (1) decreased bone formation of osteoprogenitor cells and osteoblasts and (2) increased bone resorption of osteoclasts. The patient's medical history reveals steadily decreasing bone density over the course of the previous 5 years.

The subject is treated daily with a therapeutically effective amount of a retinoid receptor agonist in a pharmaceutically effective oral dose for two months. Bone density is monitored twice a week throughout the course of therapy.

At the end of the treatment regimen, the bone density data reveal a reversal of the decrease in bone density and a statistically significant increase in bone mass as compared to the time immediately prior to the start of therapy.

The embodiments of the present invention described herein are exemplary only, and are not intended to limit the scope of the invention in any way. The invention is to be defined solely by the claims that conclude this specification.

We claim:

1. A method for treating a cartilage or bone pathology, the method comprising administering a therapeutically effective amount of a retinoid receptor agonist, or a pharmaceutically acceptable salt or ester thereof.

2. The method of claim 1, wherein said retinoid receptor agonist is an RAR receptor agonist, or a pharmaceutically acceptable salt or ester thereof.

3. The method of claim 2, wherein said RAR receptor agonist is an RARαβγ receptor agonist or a pharmaceutically acceptable salt or ester thereof.

4. The method of claim 1, wherein said pathology is a skeletal dysplasia.

5. A method for ameliorating the symptoms associated with a cartilage or bone pathology, the method comprising administering a therapeutically effective amount of a retinoid receptor agonist.

6. The method of claim 5, wherein said retinoid receptor antagonist is an RAR receptor agonist.

7. The method of claim 5, wherein said RAR receptor agonist is an RARαβγ receptor agonist.

8. A method for stimulating ossification by osteoprogenitor cells and osteoblasts, the method comprising administering a therapeutically effective amount of a retinoid receptor agonist or a pharmaceutically acceptable salt or ester thereof.

9. The method of claim 8, wherein said retinoid receptor agonist is an RAR receptor agonist, or a pharmaceutically acceptable salt or ester thereof.

10. The method of claim 8, wherein said RAR receptor agonist is an RARαβγ receptor agonist or a pharmaceutically acceptable salt or ester thereo.

11. The method of claim 10, wherein said pathology is osteoporosis.
